(19) United States
(12) Patent Application Publication
(10) Pub. No.: US 2024/0033069 A1
(43) Pub. Date: Feb. 1, 2024

(54) IN-SITU FENESTRATION DEVICES WITH ACTUATED CUTTER STRUTS

(71) Applicant: Medtronic Vascular, Inc., Santa Rosa, CA (US)

(72) Inventors: Cian WALSH, Galway (IE); Eoin J. WALSH, Galway (IE); Meriam Zahraa Waad JASSIM, Galway (IE); Emmanouil KASOTAKIS, Galway (IE)

(21) Appl. No.: 18/213,406

(22) Filed: Jun. 23, 2023

Related U.S. Application Data
(60) Provisional application No. 63/393,051, filed on Jul. 28, 2022.

(51) Int. Cl. A61F 2/07; A61F 2/954; A61B 18/14

(52) U.S. Cl. CPC A61F 2/07; A61F 2/954; A61B 18/1492; A61F 2002/061

(57) ABSTRACT

A device for creating an in-situ fenestration in a graft material of a stent graft at a fenestration site. The device includes an outer member forming a lumen and an inner member situated within the lumen of the outer member. The outer member includes one or more cutters configured to change from a delivery position to a cutting position via axial movement of the outer member and/or the inner member. The one or more cutters in the cutting position are configured to create the in-situ fenestration in the graft material of the stent graft at the fenestration site.

### CROSS-REFERENCE TO RELATED APPLICATION

This application claims the benefit of U.S. provisional application Ser. No. 63/393,051 filed Jul. 28, 2022, the disclosure of which is hereby incorporated in its entirety by reference.

### TECHNICAL FIELD

The present disclosure relates to in-situ fenestration devices with actuated cutter struts.

### BACKGROUND

In-situ fenestration (ISF) has seen limited applicability to aortic stent grafts for endovascular aneurysm repair (EVAR) and thoracic endovascular aneurysm repair (TEVAR). In-situ fenestration of aortic stent grafts can be used to maintain perfusion to blood vessels (e.g., aortic side branch arteries or peripheral arteries) located in an area excluded by a stent graft. In-situ fenestration may be used to fenestrate (e.g., create a new opening or hole) in a stent graft in-situ (e.g., in the place of the stent graft) following deployment of the stent graft within a vascular system. Application of ISF has been typically limited to removing unintentional coverage of blood vessels (e.g., arteries) upon deployment of a stent graft, but has rarely been used in elective scenarios.

### SUMMARY

In one embodiment, a device for creating an in-situ fenestration in a graft material of a stent graft at a fenestration site is disclosed. The device includes an outer member forming a lumen and an inner member situated within the lumen of the outer member. The outer member includes one or more cutters configured to change from a delivery position to a cutting position via axial movement of the outer member and/or the inner member. The one or more cutters in the cutting position are configured to create the in-situ fenestration in the graft material of the stent graft at the fenestration site.

In another embodiment, a device for creating an in-situ fenestration in a graft material of a stent graft at a fenestration site is disclosed. The device includes an outer member including a proximal end and a distal end and one or more struts extending therebetween. The one or more struts carry one or more cutters. The device also includes an inner member situated within the outer member. The one or more struts are configured to change from a delivery position to a cutting position via axial movement of the outer member and/or the inner member. The one or more cutters in the cutting position are configured to create the in-situ fenestration in the graft material of the stent graft at the fenestration site. The one or more cutters are spaced apart from the inner member in the cutting position.

In yet another embodiment, a method of deploying a device at a fenestration site to form a fenestration in a graft material of a stent graft is disclosed. The method includes delivering a distal tip of the device to the fenestration site. The method further includes penetrating the graft material of the stent graft at the fenestration site with the distal tip. The method also includes creating the fenestration at the fenestration site with one or more cutters of the device.

### BRIEF DESCRIPTION OF THE DRAWINGS

FIG. 1A depicts a partial cut away, schematic, side view of an abdominal aorta and right and left renal arteries extending therefrom where a stent graft excludes the right and left renal arteries from blood perfusion.

FIG. 1B depicts a partial cut away, schematic, side view of an aortic arch branching into a brachiocephalic artery, a left common carotid artery, and a left subclavian artery where a stent graft excludes the left subclavian artery from blood perfusion.

FIGS. 2A, 2B, and 2C depict schematic, plan views of fenestrations made to graft material bounded by a support structure and covering the ostium of a branch vessel.

FIG. 3A shows a schematic, perspective view of a cutter extending from a catheter.

FIG. 3B depicts a plan view of the cutter extending from the catheter.

FIG. 3C depicts a cross section view of the cutter and the catheter taken along line 3C-3C of FIG. 3B.

FIG. 3D depicts a schematic, side view of the cutter in a retracted position inside of the catheter.

FIG. 3E depicts a schematic, side view of a cutter in a deployed position extending from the catheter.

FIG. 4A depicts a schematic, perspective view of a support structure partially inserted into a graft material.

FIG. 4B depicts a schematic, side view of the support structure partially inserted into the graft material.

FIG. 4C depicts a schematic, perspective view of the support structure completely inserted into the graft material showing portions of the support structure extending in opposing directions relative to the graft material.

FIG. 4D depicts a schematic, perspective view of the support structure extending outward from graft material.

FIG. 5A depicts a schematic, side view of a multi-pronged centering device.

FIG. 5B depicts a schematic, end view of a multi-pronged centering device.

FIG. 5C depicts a schematic, plan view of a graft material with the distal ends of the multi-pronged device attached thereto.

FIG. 6 depicts a schematic, side view of an abdominal aorta with right and left renal arteries extending therefrom and an integrated guidewire lumen advancing therethrough.

FIG. 7 depicts a schematic, cross section view of a delivery device according to one or more embodiments.

FIG. 8A depicts a side view of a fenestration device including a sheath and a coupler at a distal end of the fenestration device.

FIG. 8B depicts a side view of a fenestration device in an exposed position where a sheath is retracted to expose a cage.

FIG. 8C depicts a plan view of a fenestration formed by a fenestration device.

FIGS. 9A through 9F depict schematic views of a fenestration device forming a fenestration within the graft material of a stent graft.

FIG. 10A depicts a side view of a cutting guidewire device in a delivery state (e.g., neutral state).

FIG. 10B depicts a cross section view of the cutting guidewire device in the delivery state taken along line 10B-10B of FIG. 10A.

FIG. 10C depicts a side view of the cutting guidewire device in a graft material penetrating state.

FIG. 10D depicts a cross section view of the cutting guidewire device in the graft material penetrating state taken along line 10D-10D of FIG. 10C.

FIG. 10E depicts a side view of the cutting guidewire device in an actuated state (e.g., a cutting state).

FIG. 10F depicts a cross section view of the cutting guidewire device in the actuated state taken along line 10F-10F of FIG. 10E.

FIG. 10G depicts schematic, cross section view of the cutting guidewire device showing a recess defined by the inner wire of the cutting guidewire device taken along line 10G-10G of FIG. 10E.

FIG. 11A depicts a schematic side view of delivering a distal tip of the cutting guidewire device through a left subclavian artery to a fenestration site.

FIG. 11B depicts a schematic side view of fenestration creation operation using struts and electrodes affixed thereto to create a fenestration at the fenestration site.

FIG. 11C depicts a schematic side view of a removal operation for removing the cutting guidewire device from the left subclavian artery and the rest of the patient's vasculature.

FIG. 12A depicts a perspective view of an alternate cutting guidewire device having blades as cutters instead of electrodes.

FIG. 12B shows a side view of a friction fit between a blade and strut where the blade is fit into an existing strut.

FIG. 12C shows a side view of a weld to secure a blade to a strut.)

FIG. 12D shows a side view of a through strut slot configured to receive a blade with a stop.

FIG. 13A is a side view showing a rivet in an uncompressed state and extending from a graft material and being delivered using a compression shaft.

FIG. 13B is a side view showing a rivet in a compressed state by retracting a rivet pull wire (e.g., pulled backwards at a handle of a delivery device to compress the rivet).

FIG. 13C is a side view showing the compression shaft and the pull wire being retracted (e.g., through a guide catheter).

FIG. 13D depicts a plan view of rivets positioned at distal ends of graft cuts to anchor graft cuts to enhance fenestration durability over time.

FIG. 14A depicts a schematic, side view of a capsule of a fenestration device tracked into position at a fenestration site of a stent graft.

FIG. 14B depicts a schematic, side view of a capsule shell being retracted to a first retracted position to reveal arms having cauterizing elements.

FIG. 14C depicts a schematic, side view of the capsule shell being retracted to a second retracted position to expand the arms into the stent wall.

FIG. 14D depicts a schematic, side view of the capsule shell being advanced back over the arms and cauterizing elements thereby disengaging the cauterizing elements.

FIG. 14E depicts a schematic, side view of the fenestration device being retracted through the guide catheter leaving a diamond shaped fenestration in the stent wall.

FIG. 15A depicts an isolated, perspective view of a stent graft fabric before a cauterization step.

FIG. 15B depicts an isolated, perspective view of a stent graft fabric after the cauterization step.

FIG. 16A depicts a perspective view of a portion of a catheter cauterization device.

FIG. 16B depicts a perspective view of 4 cauterization elements returning to their open shape memory configuration once the capsule shell is retracted.

FIG. 17A depicts a perspective view of introducing a fenestration device through a guide catheter up to a fenestration site.

FIG. 17B depicts a perspective view of screwing a distal tip of a fenestration device into the graft material at the fenestration site until the graft material drops into a slot between the distal tip and the capsule shell of the fenestration device.

FIG. 17C depicts a magnified, perspective view of the fenestration device including cauterization elements, a capsule shell, and a proximal stop.

FIG. 17D depicts a perspective view of a handle of the fenestration device including fenestration diameter markings.

FIG. 17E depicts a perspective view of the capsule shell in a proximal direction from a closed position toward an open position in response to actuation (e.g., rotation) of the handle.

FIG. 17F depicts a perspective view of cutter arms of the fenestration device in a partially open configuration.

FIG. 17G depicts a perspective view of the capsule shell in a closed position configured to capture the cutter arms.

### DETAILED DESCRIPTION

Embodiments of the present disclosure are described herein. It is to be understood, however, that the disclosed embodiments are merely examples and other embodiments can take various and alternative forms. The figures are not necessarily to scale; some features could be exaggerated or minimized to show details of particular components. Therefore, specific structural and functional details disclosed herein are not to be interpreted as limiting, but merely as a representative basis for teaching one skilled in the art to variously employ the embodiments. As those of ordinary skill in the art will understand, various features illustrated and described with reference to any one of the figures can be combined with features illustrated in one or more other figures to produce embodiments that are not explicitly illustrated or described. The combinations of features illustrated provide representative embodiments for typical applications. Various combinations and modifications of the features consistent with the teachings of this disclosure, however, could be desired for particular applications or implementations.

Directional terms used herein are made with reference to the views and orientations shown in the exemplary figures. A central axis is shown in the figures and described below. Terms such as “outer” and “inner” are relative to the central axis. For example, an “outer” surface means that the surfaces faces away from the central axis, or is outboard of another “inner” surface. Terms such as “radial,” “diameter,” “circumference,” etc. also are relative to the central axis. The terms “front,” “rear,” “upper” and “lower” designate directions in the drawings to which reference is made.

Unless otherwise indicated, for the delivery system the terms “distal” and “proximal” are used in the following description with respect to a position or direction relative to a treating clinician. “Distal” and “distally” are positions distant from or in a direction away from the clinician, and “proximal” and “proximally” are positions near or in a direction toward the clinician. For the stent-graft prosthesis, “proximal” is the portion nearer the heart by way of blood flow path while “distal” is the portion of the stent-graft further from the heart by way of blood flow path.

The following detailed description is merely exemplary in nature and is not intended to limit the invention or the application and uses of the invention. Although the description is in the context of treatment of blood vessels such as the aorta, coronary, carotid, and renal arteries, the invention may also be used in any other body passageways (e.g., aortic valves, heart ventricles, and heart walls) where it is deemed useful.

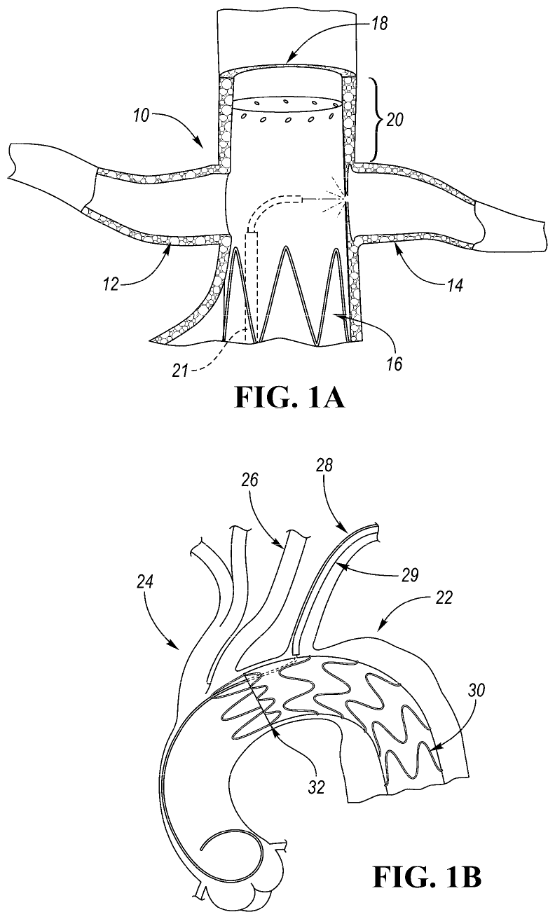

FIG. 1A depicts a partially cut away, schematic, side view of abdominal aorta 10 and right renal artery 12 and left renal artery 14 extending from abdominal aorta 10. Right and left renal arteries 12 and 14 may be referred to generally as the renal arteries. Stent graft 16 includes proximal end 18 and a distal end (not shown). Proximal end 18 of stent graft 16 lands in landing zone 20 of abdominal aorta 10. Stent graft 16 extends from landing zone 20 to exclude perfusion to right renal artery 12 and left renal artery 14. An in-situ fenestration at the exclusion areas (e.g., using laser fenestration device 21) can be used to perfuse right renal artery 12 and left renal artery 14. Perfusion may result from blood flow through the fenestration alone or through a branch stent graft inserted into the fenestration after it is created and extending into the branch artery.

FIG. 1B depicts a partial cut away, schematic, side view of aortic arch 22 branching into brachiocephalic artery 24, left common carotid artery 26, and left subclavian artery 28. Brachiocephalic artery 24, left common carotid artery 26, and left subclavian artery 28 may be referred to generally as side branch arteries. Stent graft 30 includes proximal end 32 and a distal end (not shown). Stent graft 30 extends to exclude perfusion to left subclavian artery 28. An in-situ fenestration (e.g., using laser fenestration device 29) at the exclusion area created at left subclavian artery 28 can be used to perfuse left subclavian artery 28 (e.g., via the fenestration or a later-deployed branch stent graft).

In-situ fenestration may provide a solution for implementing stent grafts with patients having hostile neck anatomy within their abdominal aorta. Current stent graft seal technology is unsuitable for many aortic anatomies. Many aortic abdominal and thoracic aortic aneurysms present either a relatively short seal zone (e.g., 0 to 10 millimeters) and/or a high degree of landing zone angulation. Examples of such anatomies include a short neck aneurysm, no neck thoraco-abdominal aneurysm, reverse conical neck, and highly angled aneurysm neck with a short landing zone inner curve. Under these circumstances, an alternative landing zone may be used that excludes perfusion to peripheral arteries (e.g., the renal arteries). In-situ fenestration may be used to open these excluded areas to permit blood perfusion. However, adequate in-situ fenestration processes and related devices/systems have not been proposed to realize the potential of in-situ fenestration in this regard.

Accordingly, clinicians (e.g., doctors or physicians) have investigated other techniques for modifying stent grafts for EVAR and TEVAR patients. The existing techniques (e.g., dedicated off-the-shelf multibranch devices, custom-made multibranch devices, clinician modified devices, and peripheral techniques) do not adequately modify stents grafts to completely address blood perfusion.

For instance, dedicated off-the-shelf multibranch devices may have low patient applicability due to variability in the anatomy of patients. The geometry to accommodate multiple branches on a dedicated branch device can be complicated to determine. Procedures to deploy these devices are complex. Branching cannulation and/or stenting can be complicated because the devices are susceptible to rotational or axial misalignment.

An alternative technology is a custom-made multibranch device. However, these devices require a significant lead time (e.g., 6 to 8 weeks) and are not available for emergent cases. Moreover, custom ordered devices may still be susceptible to axial and rotational misalignment.

Clinicians have modified stent grafts themselves before deploying the stent graft in the vascular system of the patient. Physicians can partially deploy an off-the-shelf stent graft on a sterile field and make fenestrations based on patient specific anatomy. This type of “back table” modification of an off-the-shelf stent graft may have one or more benefits. Radio frequency (RF) or thermal energy (e.g., eye cautery) may be used to clean and/or seal any frayed and/or cut fiber ends at the fenestration boundary. The size of the fenestration is customizable without post dilation, which may cause material damage. The fenestrations can be made using three-dimensional (3D) reconstructions from patient specific computed tomography (CT) scans. The fenestrations can be reinforced with sutures and/or guidewires to make a durable interface between the main stent graft and the branch stent graft. However, these procedures include unloading of the stent graft so that it can be modified with a fenestration. Reloading the stent graft is a challenge due to the low profile and high packing density of the stent graft in the radially compressed, delivery state. These modifications are typically labor and time intensive.

Techniques for providing blood flow to peripheral blood vessels used in connection with off-the-shelf stent grafts have also been proposed. Clinicians can deploy off-the shelf stent grafts in parallel with these techniques to permit blood perfusion to peripheral arteries and respective organs. Examples of these types of technologies chimneys, snorkels, and sandwich techniques. A chimney structure may be applied in the abdominal aorta and may include a renal chimney and a seal zone distal to a lower chimney. A different structure may be applied in the aortic arch where blood flows into a chimney from the aortic arch and blood flows out of the chimney into the left common carotid artery, and blood flows into a periscope from the aortic arch and blood flows out of the periscope into the left subclavian artery. Another technique is referred to as a sandwich. Blood flows into the celiac artery and superior mesenteric artery (SMA) from sandwich parallel chimneys. These techniques may have one or more of the following benefits: (1) available for emergent cases; (2) configurations can be adapted for patient-specific anatomies (e.g., ballerina techniques); and/or (3) planning using 3D reconstructions from patient specific CT scans. However, these techniques have durability concerns and potential mid or long-term occlusion risks relating to challenging hemodynamics.

Due to one or more drawbacks of the existing technologies identified above, there has been interest in developing in-situ fenestration technology that addresses one or more of the drawbacks identified above. In-situ fenestration encompasses processes in which apertures are made in a fully or partially deployed stent graft inside of a patient. Under limited circumstances, in-situ fenestration has been employed to provide perfusion in the aortic arch, the visceral segment, and the iliac arteries. In the aortic arch, in-situ fenestration can be made in a retrograde direction (e.g., outside of the stent graft) using supra-aortic access. Other anatomies may use in situ fenestration using an antegrade technique (e.g., inside the stent graft). In-situ fenestration may have one or more of the following benefits: (1) provides a multibranch solution independent of patient anatomical constraints thus providing for a larger applicability; (2) can be performed using off-the-shelf stent grafts; and/or (3) may avoid time-consuming “back-table” modification and technically challenging reloading into delivery systems.

However, current in-situ fenestration techniques suffer from one or more drawbacks. Current in-situ fenestration methods result in relatively small size apertures where aggressive post-dilation is used to accommodate a branch stent graft. Needle in-situ fenestration uses a needle to create an initial fenestration. Laser fenestration uses a laser ablation catheter having a diameter of 2.0 to 2.5 millimeters. Radio frequency (RF) ablation may also be used. One example of an RF ablation method uses a 0.035 inch powered wire. As a drawback, damage to the graft material during fenestration expansion adds to procedural variability and makes durability testing difficult. Additionally, lack of standardized protocols results in lack of consistency in fenestrations, thereby inhibiting consistent anticipation of intermediate and long-term durability.

In one or more embodiments, in-situ fenestration process and/or related devices are disclosed that at least partially addresses one or more of the following drawbacks and/or at least partially provides one or more of the following benefits. A potential drawback of existing technology is anatomical variability limiting patient applicability of dedicated off-the shelf branch devices. A potential benefit of in-situ fenestration is customization of off the shelf stent grafts that is independent of anatomical constraints. Custom devices have been proposed but take a relatively long time (e.g., 6-8 weeks) for manufacture and deliver, and may not be available for emergent cases. A potential benefit of in-situ fenestration is application to off-the-shelf devices with no manufacturing or shipping delays.

Another potential drawback relates to “back table” modification of off-the-shelf devices by clinicians. These modified devices are difficult to reload, limiting adoption of this method. In-situ modification of a stent graft occurs in-situ, and thereby eliminating the step of reloading the device into a delivery system. Custom and “back table” modified devices are susceptible to axial or rotational misalignment which can impact vessel cannulation. Fenestrations created in-situ after the deployment of a stent graft are independent of the position of the main graft.

Current in-situ fenestration procedure lack standardization in terms of initial fenestration source and post dilation procedures. A potential benefit of standardization would be the reduction or elimination of severe post dilation steps that can cause unpredictable damage to a graft material.

Current in-situ fenestration procedures may result in cut fibers and/or ripped material. These drawbacks may represent a source of procedural variability and may limit the long-term durability and seal of the fenestration and branch stent graft interface. One or more embodiments disclose a method for sealing cut fibers that help prevent continued breakdown of the fenestration and branch stent graft interface.

Current fenestration techniques start with a small initial fenestration that is aggressively post dilated to accommodate a branch graft which can result in the tearing of the graft material. Some graft materials use cutting balloons for post dilation, which may cause additional cut fibers and material damage. One or more embodiments disclose a method and/or device for forming a fenestration in-situ of a size and shape that involves little or no post dilation and/or cutting balloons.

Power sources (e.g., laser and RF ablation) for current in-situ fenestrations may create steam bubbles and generate char particles that can pose embolic risk. One or more embodiments disclose a method and/or device to allow in-situ fenestration creation while minimizing steam bubbles and char formation.

In one or more embodiments, an in-situ fenestration generation system is disclosed. The system includes a support structure configured to locate a fenestration site. The system includes a cutter configured to make cross cuts at the fenestration site. The support structure may be a support ring having a wave form profile. The cutter may be a pre-shaped electrified wire.

In one or more embodiments, a support structure positioned within a graft material of a stent graft is disclosed. The support structure is configured to reduce the likelihood of strut or suture damage during creation of an in-situ fenestration. The support structure may also improve visualization while making the fenestration. The support structure may act as a protective ring to enhance positional accuracy of crosscut(s) as described in this section. The support structure may also mitigate propagation of tears in the graft material following the crosscut(s). The support structure is configured to contain the subsequent fenestration and to create an adequate seal between a branch stent graft and the fenestration on the main stent graft.

Following placement of the support structure, an in-situ fenestration may be created using a pre-shaped electrified wire configured to cut the graft material in a cross configuration of triangular flaps of graft material (e.g., 4, 6, or 8 triangular flaps) where the base side of the graft material is connected to the remaining graft material. In one or more embodiment, triangular flaps extend 360 degrees to form a substantially circular hole. Externalization of the graft material is not needed because the flaps remain connected to the bulk graft material. The electrified wires may have a straight configuration where the wire makes a cut to the graft and then is rotated a certain number of degrees (e.g., 90 degrees) to perform a second cut (and subsequent cuts). Alternatively, the wire may be pre-shaped into a cross configuration where a single cutting pass forms all the triangular flaps.

One or more of the embodiments provides a method of creating an in-situ fenestration in which graft material does not need to be removed from a patient's vasculature. One or more methods may also reduce or minimize fraying likelihood and/or further tearing of graft material by providing reinforcement around the fenestration. The crosscut hole in the graft material can be repeatably and reliably reproduced once a pre-shaped wire for cutting is dimensioned for a branch artery.

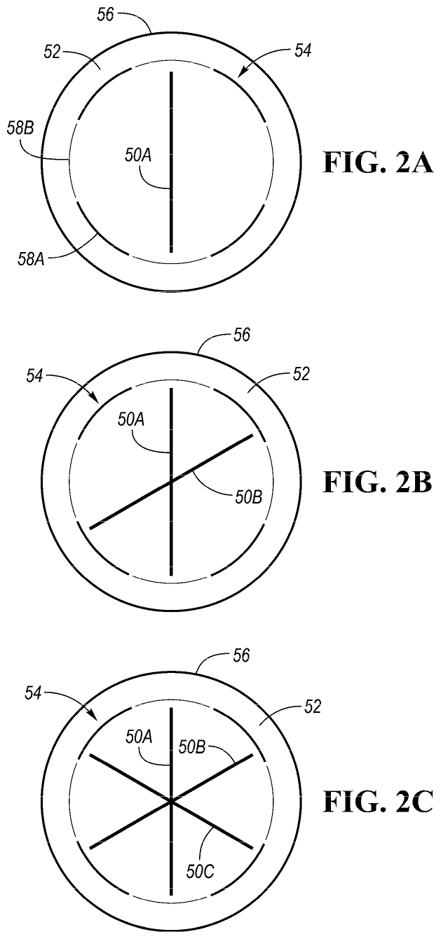

FIGS. 2A, 2B, and 2C depict schematic, plan views of fenestrations 50A, 50B, and made to graft material 52 bounded by support structure 54 and covering the ostium of branch vessel 56. In the embodiment shown, the fenestrations are formed as slits, however, other shapes may be suitable. Support structure 54 includes portions 58A extending in front of graft material 52 and away from branch vessel 56 and portions 58B extending behind graft material 52 and toward branch vessel 56. Fenestration 50A forms a slit between a 0 degree position and a 180 degree position. Fenestration 50B forms a slit between a 60 degree position and a 240 degree position. Fenestration forms a slit between a 120 degree position and a 300 degree position. As shown in FIGS. 2A, 2B, and 2C, fenestrations 50A, 50B, and 50C intersect at their apexes and collectively form 6 triangular-shaped flaps that remain connected at their bases with graft material 52. The fenestrations shown are congruent in shape. In other embodiments, the fenestrations may not be congruent and may have differences in triangular shape such that the apex angle of each of the fenestrations varies by one of the following degrees or in a range of any two of the following degrees: 1, 2, 3, 4, 5, 6, 7, 8, 9, or 10 degrees. In one or more embodiments, one or two cuts are made, leaving a 90 degree or 180 degree space between flaps. In one or more embodiments, a controlled amount of tearing of the graft material and/or under sizing of the cuts compared to the size of the stent graft are deployed in the fenestration. Fenestrations 50A, 50B, and 50C terminate on each side thereof so that there is a gap between the ends of each fenestration and support structure 54 so that the fenestrations do not interfere with the support provided by support structure 54. The gap may be any of the following dimensions or in a range of any two of the following dimensions: 0.1, 0.2, 0.3, 0.4, and 0.5 millimeters. While FIGS. 2A-2C depict a final configuration having 6 flaps, in other embodiments there may be 2, 4, or 8 flaps. As described above, the flaps in these embodiments may be congruent or non-congruent. In one or more embodiments, the support structure may be formed of a material not damaged by the cutter such that the flaps may be cut up to the support structure without causing damage. The number of cuts made may be any of the following or in a range of any two of the following: 1, 2, 3, 4, 5, 6, 7, and 8 cuts. In other embodiments, more than 8 cuts may be employed.

Support structure 54 is circular-shaped and has a perimeter that may be smaller than the perimeter of the ostium of branch vessel 56. Support structure 54 may have a wavy profile as further described herein. Support structure 54 may be a pre-shaped wire having the wavy profile to permit it to intersect graft material 52 as it is rotationally inserted. Support structure 54 may be formed of nitinol or other material having super elastic properties or shape memory properties. The distal end of support structure 54 may include an electrified tip configured to cut through the graft material.

Fenestrations 50A, 50B, and 50C may be made using an electrified wire. The electrified wire may be pre-shaped and contained within a catheter having an outer diameter suitable for transradial and/or transfemoral delivery to a branch artery. The outer diameter of the catheter may any of the following values or in a range of two of the following values: 2, 3, 4, 5, 6, 7, and 8 millimeters. The electrified, cutting wire may be formed of nitinol or stainless steel. The material forming the electrified, cutting wire may be a flexible material having relatively good electrical and heat conduction. First and second braces 104 and 106 and first and second elbows 114 and 116 may be pre-formed to hold the shape and/or position of the electrified, cutting wire.

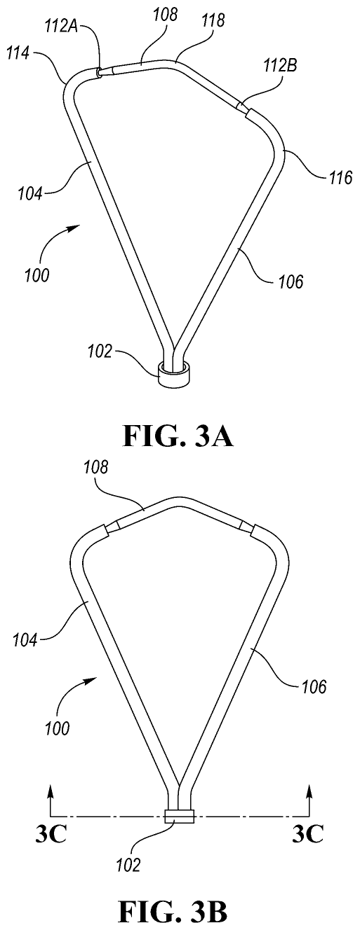

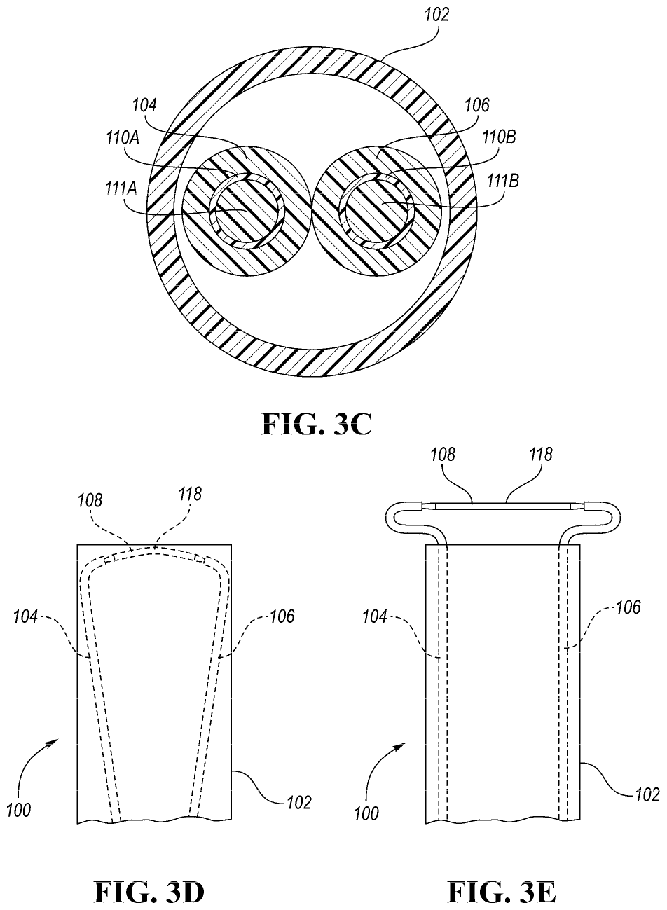

FIG. 3A shows a schematic, perspective view of cutter 100 extending from catheter 102. FIG. 3B depicts a plan view of cutter 100 extending from catheter 102. FIG. 3C depicts a cross section view of cutter 100 and catheter 102 taken along line 3C-3C of FIG. 3B. FIG. 3D depicts a schematic, side view of cutter 100 in a retracted position inside of catheter 102. FIG. 3E depicts a schematic, side view of cutter 100 in a deployed position extending from catheter 102.

Cutter 100 includes first and second braces or arms 104 and 106 and cutting element 108 extending therebetween. As shown in FIG. 3D, first and braces 104 and 106 and cutting element 108 are flexible to conform to a shape to store braces 104 and 106 and cutting element 108 within the lumen of catheter 102 in a retracted position. Upon advancing first and second braces 104 and 106 and cutting element 108, these components transition to a deployed position and the shapes as shown in FIGS. 3A, 3B, and 3E. First and second braces 104 and 106 and cutting element 108 may be pre-shaped with a shaping characteristic such that when these components transition into the deployed position, they take on the shaping characteristic. The shape may be selected so that the cutting surface does not contact the blood vessel walls during the cutting operation. After the cutting operation, first and second braces 104 and 106 and cutting element 108 are configured to retract into catheter 102 upon applying a pulling force on first and second braces 104 and 106 and cutting element 108.

Cutting element 108 may be fixedly connected at its ends to the distal ends of first and second braces 104 and 106. First and second braces 104 and 106 may be pre-formed and may configured to brace against the walls of a branch artery to define the width of the cut. In other embodiments, cutting element 108 is configured to telescope within first and second braces 106 and extend therefrom into a locked position (as shown, for example, in FIG. 3A). Cutting element 108 is connected to first wire 111A extending within first brace 104 and second wire 111B extending within second brace 106. First and second wires 111A and 111B and cutting element 108 are configured to create a circuit for electrifying cutting wire 108 upon applying power to the circuit. First and second wires 111A and 111B and cutting element 108 may form a continuous wire structure where the first and second wires have a smaller diameter than cutting element 108 to accommodate additional thickness from insulators 110A and 110B. Insulators 110A and 110B are configured to insulate first and second wires 111A and 111B from catheter 102. The continuous wire structure tapers from the larger diameter to the smaller diameter as shown in transition regions 112A and 112B. In another embodiment, the continuous wire structure may taper from a larger diameter to a smaller diameter toward peak 118 of cutting element 108.

In the deployed position, first and second braces 104 and 106 taper outward from catheter 102 to first and second elbows 114 and 116. Cutting element 108 extends from first and second elbows 114 and 116. Cutting element 108 tapers upward from elbows 114 and 116 to peak 118. Peak 118 is configured to be a leading edge for cutting fenestrations 50A, 50B, and 50C. The width between 104 and 106 at first and second elbows 114 and 116 is sized to brace against walls of a branch artery. First and second braces 104 and 106 may be electrically isolated from electrified cutting element 108 so that first and second braces 104 and 106 do not damage the walls of a branching artery. First and second braces 104 and 106 may be formed of an insulated material or include an insulating layer.

As shown in the Figures, first and second braces 104 and 106 have a circular cross-section. The diameter of first and second braces 104 and 106 may be any of the following diameters or in a range of any two of the following diameters: 0.5, 1.0, 1.5, 2.0, 2.5, 3.0, 3.5, and 4.0 millimeters. First and second wires 111A and 111B may have any of the following diameters or be in a range of any two of the following diameters: 0.4, 0.9, 1.4, 1.9, 2.4, 2.9, 3.4, and 3.9 millimeters. Catheter 102 may have any of the following diameters or be in a range of any two of the following diameters: 2, 3, 4, 5, 6, 7, and 8 millimeters.

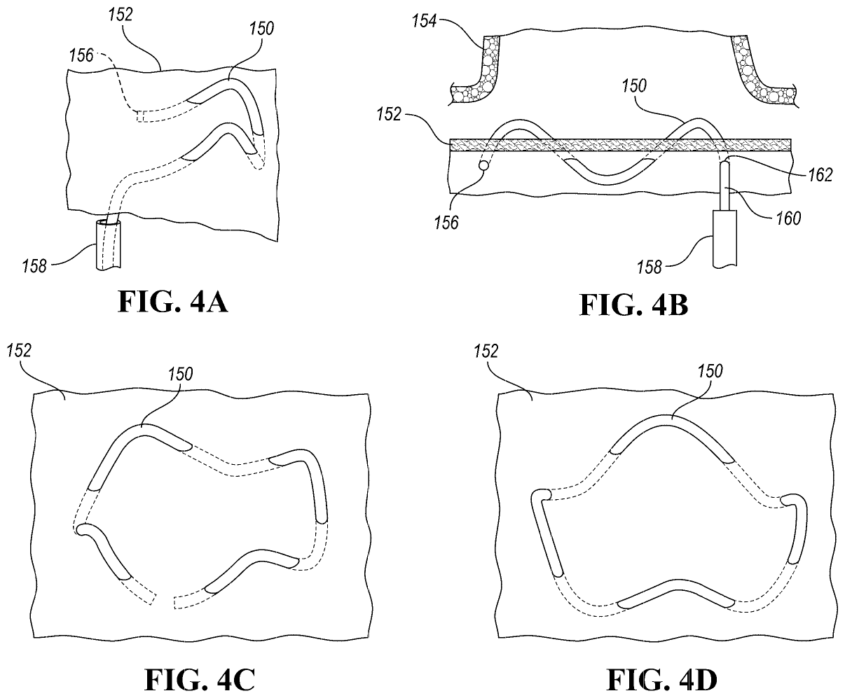

FIG. 4A depicts a schematic, perspective view of support structure 150 partially inserted into graft material 152. FIG. 4B depicts a schematic, side view of support structure 150 partially inserted into graft material 152. FIG. 4C depicts a schematic, perspective view of support structure 150 completely inserted into graft material 152 showing portions of the support structure extending in opposing directions relative to graft material 152. FIG. 4D depicts a schematic, perspective view of support structure 150 extending outward from graft material 152.

Support structure 150 has a wave form profile such that when inserted into graft material 152, alternating crests and troughs of the wave form extend from opposing surfaces of the graft material. As shown in FIG. 4C, 4 crests extend beyond one surface of the graft material and 4 troughs extend beyond the opposing surface the graft material. Other embodiments of the support structure may have other fasteners for attaching the support structure to the graft material. Non-limiting examples include hooks on one or both sides, which can be pushed into position, or electrified into place. In other embodiments, 2, 3, 5, or 6 crests/troughs extend from each surface of graft material 152, or in other embodiments, there are more crests on one side than troughs on the other side of the graft material (or vice versa). The wave form profile is self-reinforcing so that once support structure 150 is inserted into graft material 152 it tends to stay in place and not separate from graft material 152. Support structure 150 forms an overall circular shape following the overall shape of an ostium of branch vessel 154. The amplitude between adjacent waves of the wave form may be any of the following values or in a range of any two of the following values: 1, 2, 3, 4, 5, 6, 7, 8, 9, and 10 millimeters.

Support structure 150 may include an electrified tip 156 configured to easily cut through graft material 152. Support structure 150 may define a lumen for receiving a conductor for electrifying tip 156. Electrifying tip 156 may be electrified by having a surface that is free from insulation and using an external generator adding energy to the wire. The support structure may be connected to the wire in the catheter to create a connection and is decoupled during deployment. In another embodiment, the tip of support structure may be sharpened or pointed such that it can cut through graft material without electrification. In another embodiment, the tip may be vibrated at a high frequency (e.g., ultrasonic) to help it pierce the graft material. Support structure 150 may be formed of nitinol or other material having super elastic properties or shape memory material. Support structure 150 may be preloaded into catheter 158 in a retracted position where support structure 150 is partially or completely disposed within the lumen of catheter 158. Delivery wire 160 may be advanced after tip 156 is electrified to sew/weave support structure 150 into graft material 152. The material forming support structure 150 may have a shape memory characteristic such that as the support structure 150 is advanced distal to catheter 150 it transitions into the ring-like structure shown in the Figures. Electrified tip 156 may form an opening in the graft material when the two make contact, allowing the support structure 150 to continue to be advanced, thereby self-sewing/weaving itself into the graft material. The shape memory characteristic also aids in positioning support structure 150 into graft material 152. The shape memory characteristic helps guide the distal tip of catheter 158 into a proper deployment position. After deployment of support structure 150 into graft material 152, support structure 150 and delivery wire 160 are configured to separate from each other at seam 162. The support structure may be attached via a paddle and pocket connection, utilizing a catheter as a way of holding the two together. Alternatively, a pull wire pin connection may be used where a pull wire removes a pin that connects the support structure to the rest of the energy transferring wire. The support structure may be restrained by its own radial force within the catheter at a bulky section thereof that is configured to be the last to exit the catheter where the inner surface of the catheter transfers energy directly to the support structure.

One or more embodiments disclose mechanisms and/or operations for centering the support structure and/or cutting operation. A hollow balloon may be used to center the support structure and/or cutting operation. The hollow balloon may be configured to locate the support structure or cutter centrally to enable blood flow through the patient's vasculature (e.g., to the brain). Prongs on the hollow balloon may be configured to provide anchoring to the support structure to allow it to penetrate through the graft material. Alternatively, a guidewire (e.g., a stiff guidewire) may be placed at the center of the desired location of the support structure and/or fenestrations. The guidewire is configured to provide a path for the cutter to advance along.

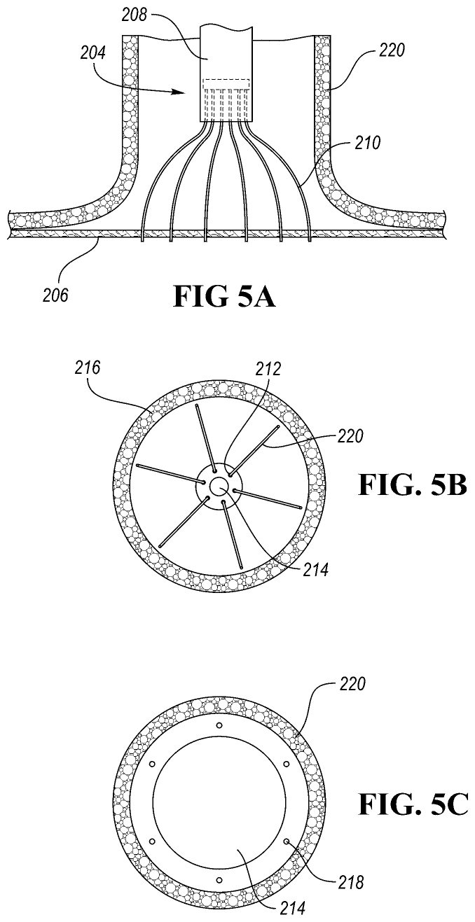

FIG. 5A depicts a schematic, side view of multi-pronged centering device 204. FIG. 5B depicts a schematic, end view of multi-pronged centering device 204. FIG. 5C depicts a schematic, plan view of graft material 206 with the distal ends of multi-pronged center device 204 attached thereto. Device 204 includes catheter 208 and legs 210 extending distally therefrom. Catheter 208 may be delivered through a branch vessel (e.g., as shown in FIG. 5A).

While 6 legs are shown in the figures, there may be less or more legs (e.g., 4, 5, 7, or 8) depending on the implementation. Legs 210 are configured to be advanced from a retracted, stowed position to an advanced, deployed position. The deployed position is shown in FIG. 5A. As shown in FIG. 5A, legs 210 are configured to be disposed in apertures 212 defined by catheter 208. Catheter 208 defines lumen 214 configured to permit tracking of a cutter or support ring in retracted positions therethrough. The distal ends of legs 210 are configured to attach to or engage the graft material 216 at attachment/engagement points 218. The attachment/engagement points may form a substantially or entirely circular profile having a perimeter less than (e.g., slightly less than) the perimeter of branch vessel 220.

Legs 210 may be formed of nitinol or other material having super elastic properties. Legs 210 have a shape memory characteristic such that legs 210 spread out as they extend past the distal end of catheter 208 and then push through graft material 216 to form attachment/engagement points 218. Legs 210 are configured to anchor graft material 216 at attachment/engagement points 218 and centers cutter and/or support structure relative to attachment points 218 as cutter and/or support structure advances past distal end of catheter 208. The cutter and/or support structure are configured to be placed at the center of legs 210 to provide accurate placement.

The support structure delivery and/or cutting operation may be carried out using transradial/transfemoral access for ease of alignment of the in-situ fenestration with a branch artery. A centering device may also be used to deliver a wire through the graft from the branch side of the vessel. This wire may be used as a guide the cutter tool uses from a transfemoral access route to position itself centrally within the branch. A catheter containing a pre-shaped support structure (e.g., wire ring) may be tracked to a fenestration site and subsequently deployed at the fenestration site. A delivery wire attached to the support structure may be detached once the support structure is deployed and then the delivery wire may be removed from the catheter. A cutter can then be inserted into and tracked along the catheter (or a separate cutter catheter may be used). The cutter is then deployed through the distal tip of the catheter by electrifying the tip and cutting crosscut holes/slits into the graft material. Pre-shaped wires can be used for the cutter and/or support structure. The pre-shaped wires can be crimped into relatively smaller catheter sizes, while retaining its unconstrained shape, which makes a 6 to 12 Fr catheter for transradial/transfemoral access suitable. The support ring and wire cutter may have different delivery systems. The support structure is configured to provide visual alignment to help mitigate the likelihood of cutting into a vessel wall. The support structure may also mitigate the effects of a curve that may exist at the junction between the branched artery and aorta which may affect positioning of the in-situ fenestration.

One or more embodiments of a combination of support structure and cutter provide one or more of the following benefits. One or more embodiments may minimize fraying of graft material. One or more embodiments may eliminate externalizing cut graft material. One or more embodiments may provide visual alignment from the support structure. This visualization may increase in-situ positioning accuracy.

In one or more embodiments, an in-situ fenestration device is disclosed. The in-situ fenestration device may include a sheath and a coupler at a distal end of the in-situ fenestration device. The in-situ fenestration device may further include a cage in a crimpled position when contained within the sheath and in an expanded position when the sheath is moved in a proximal direction to form a gap between the coupler and a distal end of the sheath. The in-situ fenestration device includes a wire connected to the coupler and configured to change a diameter of the cage upon applying a pulling force on the wire.

One or more embodiments are directed to an in-situ fenestration system configured to create fenestrations of varying diameter depending on the size of the ostium of a branch vessel. The in-situ fenestration system is also configured to maneuver through the vasculature of a patient. In one or more embodiments, the in-situ fenestration system is configured to achieve relatively high angulation for relatively complex anatomies and stability. One or more embodiments may use steerable catheter features and/or flexible catheter features. The system may include a pull wire based one-plane steering mechanism configured to add more stability to a fenestration. The system may include a braided shaft configured to provide torquing and translation. The system may access and maintain fenestration access by steering and using ostial beacons with integrated guidewire/penetrating wire. In one or more embodiments, increased repeatability of fenestration is provided via steering features and/or an adjustable diameter. The system may also provide an easily adjustable diameter of fenestration.

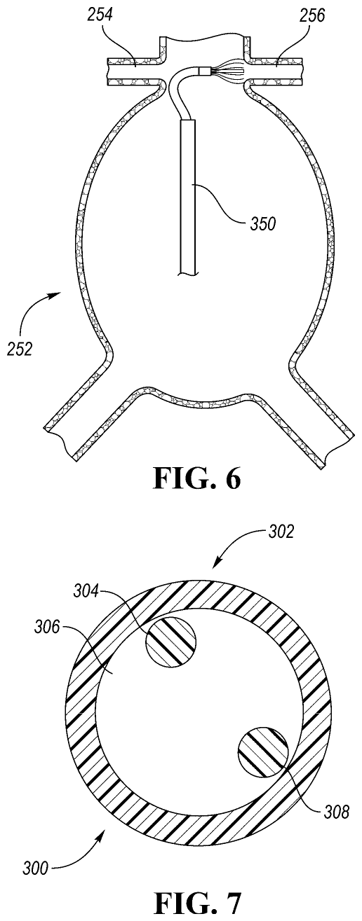

The in-situ fenestration system may use a transfemoral access pathway. Imaging techniques, such as computed tomography (CT) scans and ultrasound imaging, may be used to determine a size and location of a fenestration. FIG. 6 depicts a schematic, side view of abdominal aorta 252 with right and left renal arteries 254 and 256 extending therefrom and fenestration device 350 (further described below) advancing through abdominal aorta 252. The alignment and location of the branch vessel (e.g., left renal artery 256) may use an ostial beacon in connection with ultrasound. The ostial beacon may be disposed on the distal end of an intravascular ultrasound (IVUS) device. In one or more embodiments, the ostial beacon is aligned with ultrasound to obtain access to a fenestration site. Fenestration device 350 includes an integrated guidewire lumen with an inner penetrating guidewire configured to track adjacent to the ostial beacon. Once the integrated guidewire lumen is tracked, the ostial beacon can be removed.

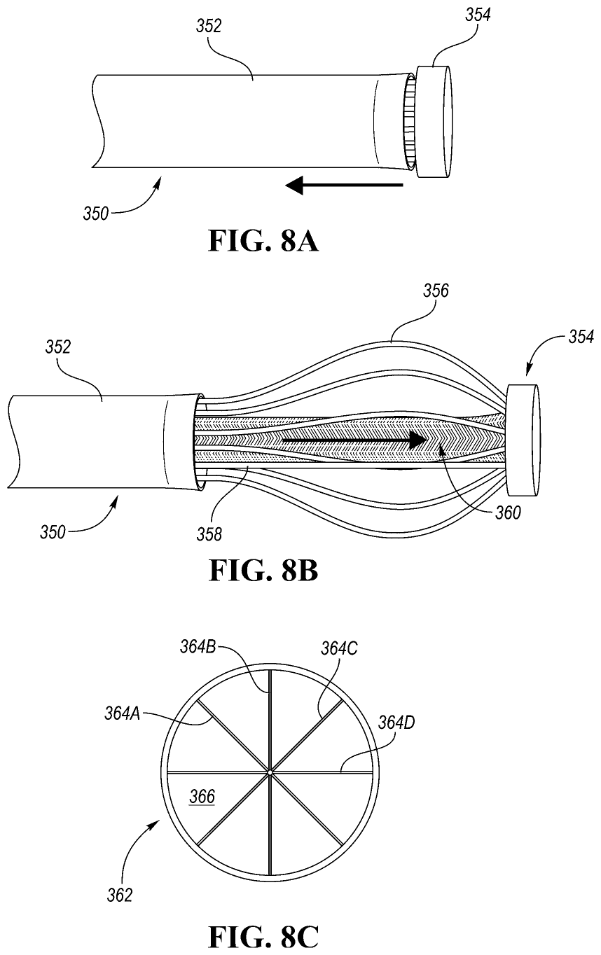

The penetrating guidewire is tracked into the branch vessel and the catheter is tracked over the penetrating wire to the ostium of the branch guidewire. The dilator is tracked over the guidewire and is configured to create an initial fenestration via RF. Orthogonal flex may be added to the inner catheter to permit creation of precise and stable fenestrations by maintaining stability during the creation of the fenestration. As a step in creating the fenestration, a coupler may be pulled back to deploy a distal cage assembly via a handle. A knob located on a delivery device handle may be rotated to pull on a wire connected to a coupler (e.g., as shown in FIGS. 8A and 8B) to adjust a diameter of the cage (e.g., to a diameter determined during pre-screening). RF can then be applied to the cage, which can be slowly inserted into the ostium of the branch vessel to create the fenestration. The cut graft material may be pushed out naturally from blood flow via the cage or removed via RF. At this point, the fenestration catheter may be removed, and the branch stent graft deployed.

FIG. 7 depicts a schematic, cross section view of delivery device 300. Delivery device 300 includes outer sheath 302, inner steering lumen and wire 304, inner member 306, and diameter adjustment wire 308. Outer sheath 302 is configured to cover a cage (as described herein) during tracking. The coupling for the cage is atraumatic. Inner member 306 is configured to flex for positioning up to a maximum degree of flex (e.g., a 90 degree angle) to align with a fenestration site on the graft material. At a 90 degree angle, the distal tip of the inner member 306 is normal to the fenestration site. A knob or slider on delivery device 300 may be used to activate the bend in inner member 306. Inner member 306 may have a braided shaft to provide a torque. Dual lumens may be utilized in one or more embodiments. The first lumen may be configured to steer the catheter. The second lumen may be configured to adjust a cage diameter (as further described below). The handle of the delivery device may have an indicator to select a size of the fenestration. The handle may actuate a pulling force on a central wire that increases a diameter of a fenestration.

FIG. 8A depicts a side view of fenestration device 350 including sheath 352 and coupler 354 at a distal end of fenestration device 350. Coupler 354 may be formed of a metal material configured to be easily visualized in Fluoro (e.g., a radiopaque material). FIG. 8A shows fenestration device 350 in a crimped position where sheath 352 is proximate to coupler 354. FIG. 8B depicts a side view of fenestration device 350 in an exposed position where sheath 352 is retracted to expose cage 356. Cage 356 may be formed of a pre-shaped metal material (e.g., nitinol) such that when sheath 352 is retracted cage 356 transitions from a crimped state to an expanded state. Wire 358 is attached distally to coupler 354 on the distal end of fenestration device 350 and proximally to a handle (not shown) of fenestration device. Wire 358 may be contained within the lumen of sheath 352 or in a separate lumen. Applying a pulling force to wire 358 via handle of fenestration device 350 increases the diameter of cage 356. Fenestration device 350 also includes dilator 360 running through the center of the device over the guidewire and having an RF tip configured to create an initial center aperture of the fenestration. Once the initial center aperture is formed, the penetrating guidewire can be advanced through the initial center aperture and used to advance fenestration device 350.

FIG. 8C depicts a plan view of fenestration 362 formed by fenestration device 350. Fenestration 362 includes cross cuts 364A, 364B, 364C, and 364D created as cage 356 advances through graft material at the fenestration site. Cross cuts 364A, 364B, 364C, and 364D form triangular flaps 366 that are attached to the graft material at perimeter 368 of fenestration 362. Flaps 366 are configured to flare outward in the direction of blood flow into the branch artery. As shown in FIG. 8C, there are 8 triangular flaps that correspond to 8 wires comprising cage 356. In other embodiments, there may be 3, 4, 6, or 10 triangular flaps and corresponding wires of the cage. As shown in FIG. 8C, the triangular flaps are congruent in shape. In other embodiments, the fenestrations may not be congruent and may have differences in triangular shape such that the apex angle of each of the fenestrations varies by one of the following degrees or in a range of any two of the following degrees: 1, 2, 3, 4, 5, 6, 7, 8, 9, or 10 degrees.

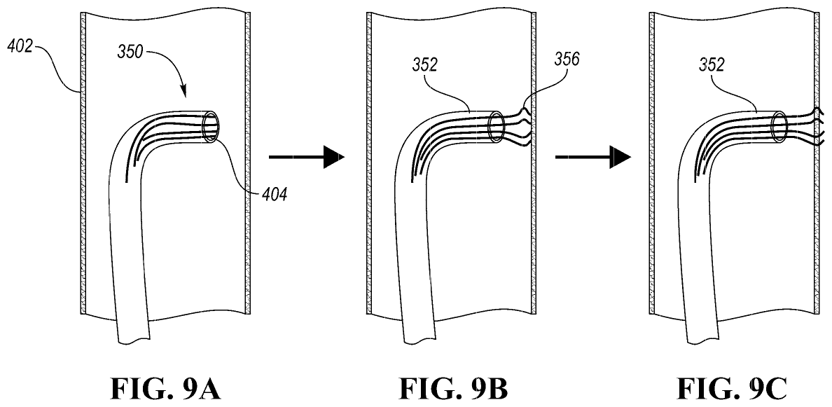

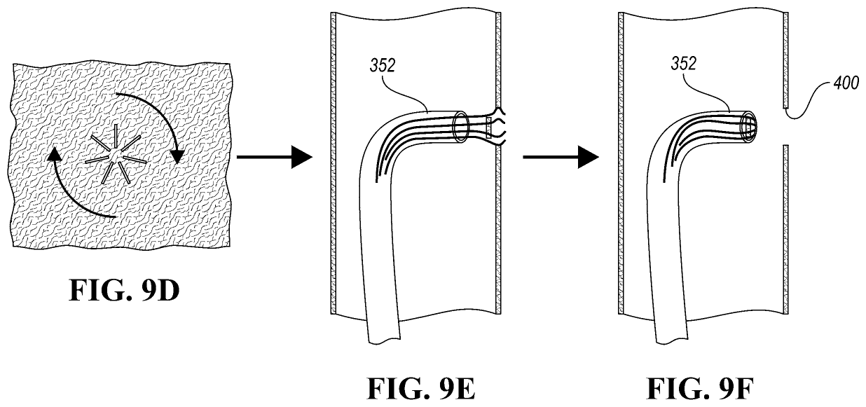

FIGS. 9A through 9F depict schematic views of fenestration device 350 forming fenestration 400 within the graft material of stent graft 402. As shown in FIG. 9A, distal end 404 of fenestration device 350 is facing a fenestration site on the graft material of stent graft 402. As shown in FIG. 9B, cage 356 is shown in the expanded state by retracting sheath 352. The diameter of cage 356 may be adjusted using wire 358 as described above. Once the diameter of cage 356 is set, relatively short bursts of RF energy may be used to cut through the graft material at the fenestration site using the wires comprising cage 356 as shown in FIG. 9C. An RF energy technique for transseptal punctures may be used to apply RF energy to the wires comprising cage 356. Each relatively short burst of RF energy may be at a power level and a duration. The power level may be any of the following or in a range of any two of the following: 8, 9, 10, 11, and 12 watts. The duration may be any of the following or in a range of any two of the following: 1, 2, 3, 4, 5, 6, 7, 8, 9, and 10 seconds. The RF energy cause a vibrational oscillation of cage 356 configured to cut the graft material.

As shown in FIG. 9D, rotational movement of cage 356 may be used to create the fenestration. The rotational movement may be isolated from the outer sheath which uses its own lumen utilizing previously discussed tracking and steering mechanisms. The amount of sheath retraction may be used to define the diameter of cage 356, and therefore, the diameter of the fenestration. The angle of claw tip of dilator 360 is configured to maintain cut graft material within the inside of cage 356. At this point, sheath 352 may be advanced toward coupler 354 to transition cage 356 back into the crimped state and trapping the cut graft material. Trapping of the cut graft material may not be necessary in some embodiments, such as the fenestration shown in FIG. 8C where flap remain attached to the graft material. However, in some embodiments, it may be beneficial to remove all cut graft material using the rotational movement, as described.

One or more embodiments disclose in-situ fenestration systems configured to create a controlled fenestration in a graft material. The in-situ fenestration systems of one or more embodiments are compatible with CT and/or ultrasound for procedural guidance and/or imaging. The in-situ fenestration systems of one or more embodiments provides reliable steering and controlling mechanisms that may reduce procedural times and/or provide consistent results. The in-situ fenestration systems can conform to a variety of graft and vessel anatomies.

In one or more embodiments, a cutting guidewire device for creating an in-situ fenestration in a graft material of a stent graft is disclosed. The cutting guidewire device may include an outer member and an inner member translatable within the outer member. The outer member includes one or more cutters configured to translate from a delivery position to a cutting position via axial movement of the outer member relative to the inner member.

One or more embodiments disclose an actuated cutting guidewire device configured to create an in-situ fenestration in graft material of a vascular stent graft. In one or more embodiments, a single device is used to create a thermal cut that does not damage the device and creates a durable fenestration followed by a branch stent graft placement.

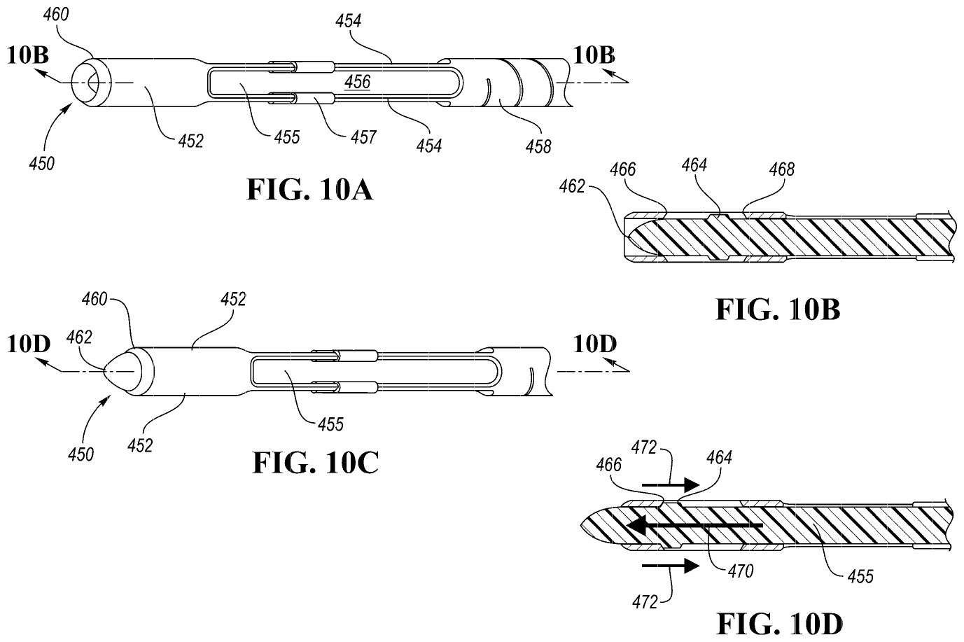

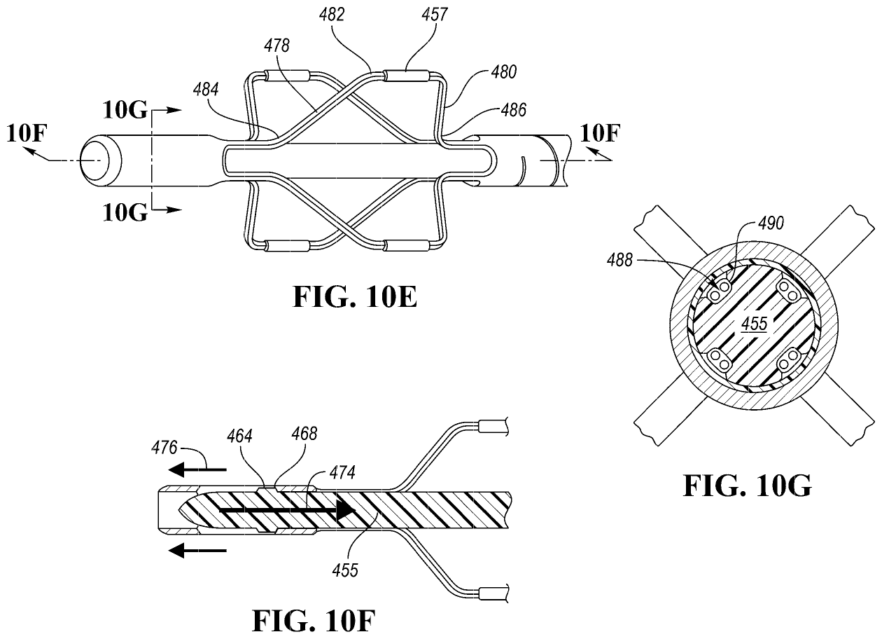

FIG. 10A depicts a side view of cutting guidewire device 450 in a delivery state (e.g., neutral state). FIG. 10B depicts a cross section view of cutting guidewire device 450 in the delivery state taken along line 10B-10B of FIG. 10A. Cutting guidewire device 450 includes outer member 452 including struts 454 defining axial windows 456 therebetween. While cutting guidewire device 450 is shown with 4 struts, in other embodiments, there may be more or less struts, e.g., 3, 5, 6, 7, or 8 struts. Cutting guidewire device 450 also includes inner wire 455 situated within the lumen defined by outer member 452. The outer diameter of inner wire 455 may be any of the following diameters or in a range of any two of the following diameters: 0.018, 0.019, 0.020, 0.021, and 0.022 inches. The outer diameter of outer member 452 may be any of the following diameters or in a range of any two of the following diameters: 0.023, 0.024, 0.025, 0.026, and 0.027 inches. Struts include electrode 457 (e.g., radiopaque electrodes). Cutting guidewire device 450 also includes proximal portion 458. Cutting guidewire device 450 may be formed using laser-cutting, milling, or welding process. During the manufacturing process, struts 454 may be formed by removing the material that form the axial windows 456.

Outer member 452 may be a laser cut hypotube. Outer member 452 may be formed of nitinol or other material having super elastic properties. Distal tip 460 of outer member 452 may be formed of a radiopaque material. As shown in FIG. 10A, proximal portion 458 of outer member 452 includes a transverse cutting pattern configured to facilitate flexing and deliverability. Axial windows 456 may be cut distally to facilitate actuation of struts 454. Axial windows 456 may be cut with a laser.

Inner wire 455 includes distal tip 462 configured to pierce the graft material at a fenestration site. The distal tip may be a sharpened tip that punches through the graft material at the fenestration site. Alternatively, distal tip 462 may cut through the graft material via high frequency vibration (e.g., ultrasound) or via RF or heat energy. Inner wire 455 also includes retaining shoulder 464 configured to translate between distal stop 466 and proximal stop 468. When retaining shoulder 464 contacts distal stop 466, inner wire 455 is in an advanced position relative to outer member 452. In the advanced position, distal tip 462 is exposed beyond distal tip 460 of outer member 452 and is configured to pierce the graft material at the fenestration site to create an initial fenestration that may anchor cutting guidewire device 450 for a subsequent cutting operation. When retaining shoulder 464 contacts proximal stop 468, inner wire 455 is in a retracted position relative to outer member 452. When cutting guidewire device 450 is advancing through the vasculature of the patient to reach the fenestration site, inner wire 455 may be in the delivery or retracted position so that piercing distal tip 462 is retracted into outer member 452. In this retracted position, piercing distal tip 462 does not contact the patient's tissue thereby mitigating the likelihood of any damage to the tissue.

FIG. 10C depicts a side view of cutting guidewire device 450 in a graft material penetrating state. FIG. 10D depicts a cross section view of cutting guidewire device 450 in the graft material state taken about line 10D-10D of FIG. 10C. In the graft material penetrating state, distal tip 462 of inner wire 455 extends beyond distal tip 460 of outer member 452 such that distal tip 462 is exposed to pierce the graft material. Cutting guidewire device 450 may be oriented such that distal tip 462 faces the graft material during the graft material penetrating state. Distal tip 462 of inner wire 455 may maintain its extension beyond distal tip 460 of outer member 452 by a selectable locking mechanism (not shown). The locking mechanism is configured to maintain the inner member in a state of compression and the outer member in a tensile state, thereby maintaining the distal tip of the inner wire in its penetrating state. As shown by arrow 470, inner wire 455 may translate in a distal direction to achieve the penetrating state. As shown by arrows 472, outer member 452 may translate in a proximal direction to achieve the penetrating state. In one or more embodiments, a combination of movement in the distal and proximal directions may be used to achieve the penetrating state. As shown in FIG. 10D, retaining shoulder 464 contacts distal stop 466 in the penetrating state as shown.

FIG. 10E depicts a side view of cutting guidewire device 450 in an actuated state (e.g., a cutting state). FIG. 10F depicts a cross section view of cutting guidewire device 450 in the actuated state taken along line 10F-10F of FIG. 10E. As shown by arrow 474, inner wire 455 may translate in a proximal direction to achieve the cutting state. As shown by arrows 476, outer member 452 may translate in a distal direction to achieve the cutting state. In one or more embodiments, a combination of movement in the distal and proximal directions may be used to achieve the cutting state. As shown in FIG. 10F, retaining shoulder 464 contacts proximal stop 468 in the cutting state.

In the cutting state, struts 454 transition from a neutral position where struts 454 extend linearly between distal end of outer member 452 and proximal portion 458 of outer member 452 to a cutting position where struts 454 have tapered portions 478 and 480 and raised central portion 482. Tapered portions 478 and 480 bend at elbows 484 and 486, respectively. The distance between distal end of outer member 452 and proximal portion 458 of outer member 452 in the cutting position is less than the distance in the delivery position. The distance in the delivery position may be any of the following distances or in a range of any two of the following distances: 10, 15, 20, 25, 30, 35, 40, 45, 50, 55, and 60 millimeters. The distance in the cutting position may be any of the following distances or in a range of any two of the following distances: 6, 10, 15, 20, 25, 30, 35, 40, 45, and 50 millimeters. The reduction in distance may be achieved using a selectable locking mechanism where upon force being applied transitions the struts into the cutting position instead of translating the entire outer member 452. Struts 454 may have a shape memory characteristic such that when this force is applied the struts 454 take on the shape shown in FIG. 10E.

Struts 454 include electrodes 457 affixed thereto. As shown in FIGS. 10C and 10E, electrodes 457 are centrally located along the length of struts 454. In other embodiments, electrodes 457 may be located in a distal position along the length of struts 454 or may be located in a proximal position along the length of struts 454, or a combination of central, distal, and proximal positions to achieve certain cutting characteristics for cutting a fenestration at a fenestration site. The benefits of locating the electrodes proximally or distally reduces the likelihood of an electrode contacting and/or damaging the vessel wall. Electrodes 457 may be formed of a radiopaque material configured to enhance visualization during the in-situ fenestration procedure.

Electrodes 457 are electrically connected to power cables 488 (e.g., multistrand power cables) as shown in FIG. 10G. FIG. 10G depicts a schematic, cross section view of cutting guidewire device 450 showing recess 490 defined by inner wire 455 taken along line 10G-10G of FIG. 10E. Power cables 488 are run through channels 490 within inner wire 455 and electrically connect to electrodes 457. One power cable 488 may connect to one electrode 457, therefore providing four power cables for four electrodes. In other embodiments, a single power cable may be run proximate to electrodes 457 and then connect to electrodes 457 in series or in parallel depending on packaging characteristics. Power cables 488 provide power to electrodes 457 to cut a fenestration by creating flaps at the fenestration site or by creating an aperture by rotating struts 454 as they are advanced during the cutting operation.

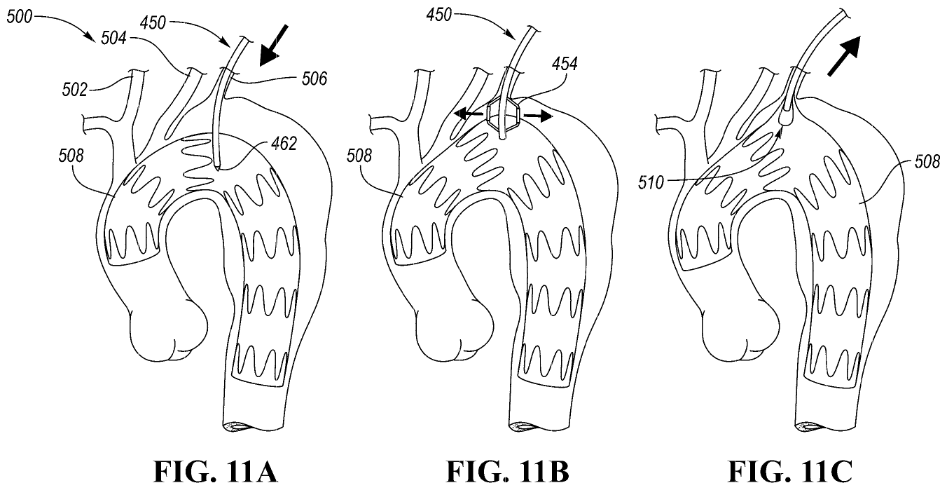

FIGS. 11A, 11B, and 11C depict schematic, side views of aortic arch 500 in connection with a procedure to deliver cutting guidewire device 450 to a fenestration site. Aortic arch 500 branches into brachiocephalic artery 502, left common carotid artery 504, and left subclavian artery 506.

FIG. 11A depicts delivering the distal tip of cutting guidewire device 450 through left subclavian artery 506 to the fenestration site. While access for cutting guidewire device 450 through the left subclavian artery is shown in FIG. 11A, it is also contemplated that femoral access may be used. Using fluoroscopy, ultrasound (e.g., IVUS), or other suitable imaging or visualization method, distal tip 462 of inner member 455 is located to stent graft 508. Once the clinician is satisfied with the location of distal tip 462, the graft material penetrating state of cutting guidewire device 450 is engaged by compressing inner wire 455 and tensioning outer member 452, thereby resulting in distal tip 462 protruding distally from outer member 452. In one or more embodiments, distal tip 462 has a sharp piercing distal end, which is pushed against the graft material of stent graft 508 to pierce the graft material to create an initial fenestration at the fenestration site. A guide catheter and/or balloon catheter may be used for additional support and/or to facilitate locating distal tip 462 or centering device 450 in the branch artery.

FIG. 11B depicts a fenestration creation operation using struts 454 and electrodes 457 of affixed thereto to create a fenestration at the fenestration site. Cutting guidewire device 450 is further advanced to a position where the initial fenestration aligns with electrodes 457 of struts 454. Once alignment is achieved, inner wire 455 is tensioned to retract the sharp piercing distal tip to prevent unintended perforations, and at the same time, outer member 452 is compressed, which causes struts 454 to compress outward against the graft material surrounding the initial fenestration. Electrodes 457 are then briefly powered (e.g., 1 to 10 seconds), thereby creating a fenestration with a number of slits equal to the number of electrodes and extending away from the initial fenestration along the graft material to facilitate subsequent placement of branch stent graft. The slits may be equally spaced. While cutting guidewire device 450 is shown with 4 struts/electrodes, in other embodiments, there may be more or less struts, e.g., 3, 5, 6, 7, or 8 struts, to create more or less slits as part of the fenestration.

FIG. 11C depicts a removal operation for removing cutting guidewire device 450 from left subclavian artery 506 and the rest of the patient's vasculature. Following creation of fenestration 510, cutting guidewire device 450 is removed from stent graft 508 and left subclavian artery 506, and a branch stent graft is delivered using a delivery technique. When the inner wire is advanced distally and compression is no longer applied, the struts return to their previous shape. The inner wire may be briefly advanced distally against the distal stop to ensure a return to a smaller diameter shape.

One or more embodiments have one or more of the following benefits. The piercing actuated material cutting guidewire may be used in conjunction with other alternate concepts. One or more embodiments provide a safety mechanism where piercing inner member (e.g., core wire) is only active during crossing (e.g., penetrating) of the graft material, and it otherwise retracts in the outer member preventing accidental penetration or dissection of vessels. Due to pre-loading the cutting guidewire device for expansion of the struts prior to application of heat, minimal heat energy is delivered to the vessel due to the short length of time of heat application, thereby reducing the likelihood of tissue damage. The fenestration cuts may be melted or heated to facilitate fenestration durability.

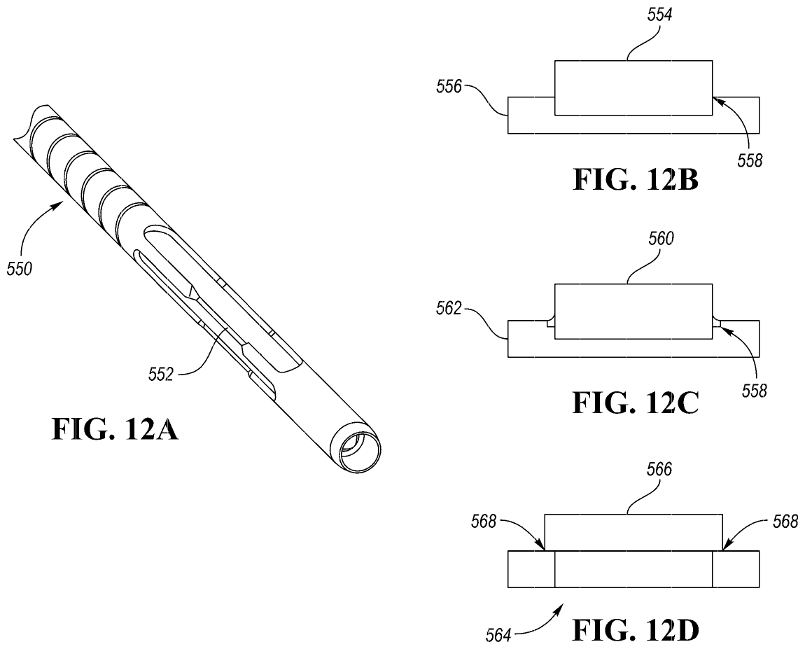

FIG. 12A depicts a perspective view of alternate cutting guidewire device 550 having blades 552 instead of electrodes 457 as cutters. FIG. 12A depicts blades 552 shaped out of expanding struts. Alternatively, the blades may be added to existing struts as shown in FIGS. 12B, 12C, and 12D. FIG. 12B shows a friction fit between blade 554 and strut 556. FIG. 12C shows weld 558 to secure blade 560 to strut 562. In one embodiment, the weld melts both the strut and blade material together. In an alternate embodiment, only the material is melted or flowed onto or into the strut material for a physical attachment or fit. FIG. 12D shows through strut slot 564 configured to receive blade 566 with stop 568. The strut may be formed of nitinol and the blade may be formed of a stainless steel. In embodiments with blades, a protective outer sheath may be included to cover the blades during tracking to/from the fenestration site. The sheath may be retracted to expose the blades when the fenestration is to be formed. A sheath may be used in any embodiment of the device; however, the retracting inner wire may make it unnecessary in some embodiments.

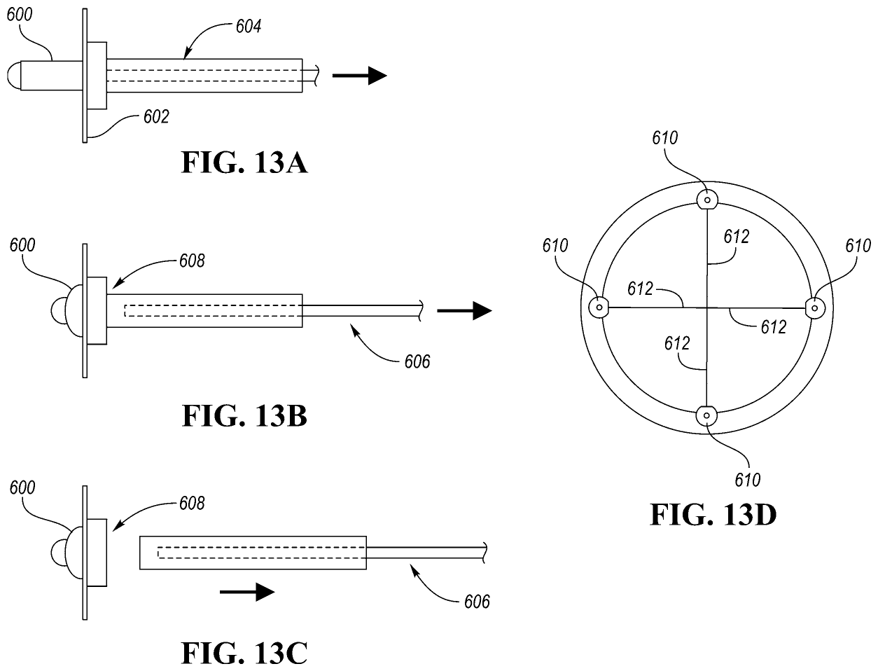

In an alternate embodiment of a piercing cutting guidewire device, metallic rivets may be used to stabilize graft cuts and to provide fenestration durability over time. The rivets may be used with a thermal (e.g., electrode 457) embodiment or an alternative mechanical embodiment. FIG. 13A is a side view showing rivet 600 in an uncompressed state and extending from graft material 602 and being delivered using compression shaft 604. FIG. 13B is a side view showing rivet 600 in a compressed state by retracting rivet pull wire 606 (e.g., pulled in a proximal direction at a handle of a delivery device to compress rivet 600), thereby causing pull wire shear 608. FIG. 13C is a side view showing compression shaft 604 and pull wire 606 being retracted (e.g., through a guide catheter). The flat section of the rivet geometry may be changed to permit the rivet to use a cutting strut as a guide rail to locate a riveting position. FIG. 13D depicts a plan view of rivets 610 positioned at distal ends of graft cuts 612 to anchor the graft cuts to enhance fenestration durability over time.

In one or more embodiments, an in-situ fenestration device is disclosed that is configured to create a fenestration using radially spaced wire cutting elements. The device includes a sheath (e.g., a capsule shell) and a plurality of arms supporting the radially spaced wire cutting elements. The plurality of arms are configured in a compressed state when the sheath is in a closed position. The plurality of arms are configured in an expanded state when the sheath is in an open position (e.g., a partially open position). In the expanded state, the radially spaced cutting elements are configured to cut a fenestration in a graft material of a stent graft.

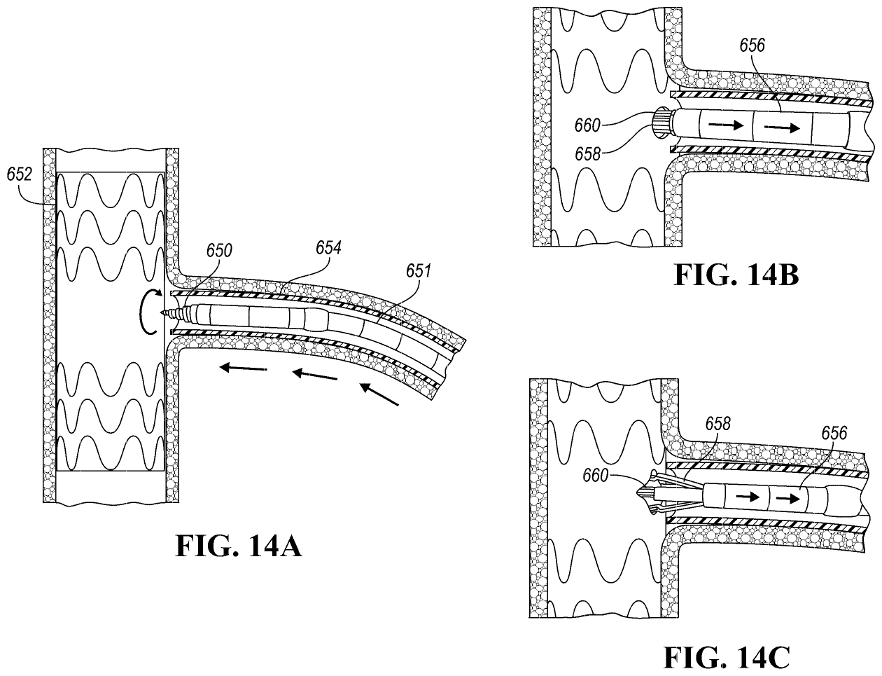

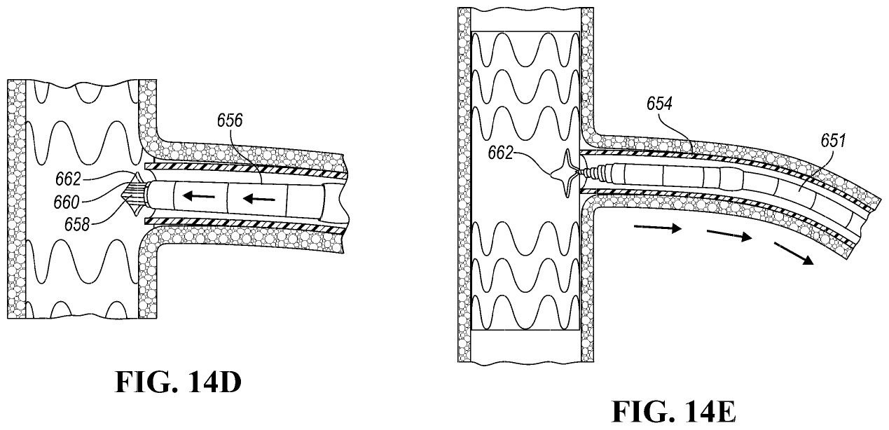

An expanding hot wire cauterizer is disclosed. The expanding hot wire cauterizer is configured to make an in-situ fenestration in a graft material of a stent graft. FIG. 14A depicts a schematic, side view of capsule 650 of fenestration device 651 tracked into position at a fenestration site of stent graft 652. Capsule 650 is tracked into position through guide catheter 654. Capsule 650 may be tracked along a guide wire extending through a pilot hole in the graft material. When contact is made with the stent wall of stent graft 652 at the fenestration site, fenestration device 651 (including, or independent of, capsule 650) is rotated to form an initial hole at the fenestration site. The initial hole increases in diameter as capsule 650 is rotated.

FIG. 14B depicts a schematic, side view of capsule shell 656 being retracted to a first retracted position to reveal struts 658 having cauterizing elements 660. The struts may be formed of nitinol or a different super elastic material. As shown in FIG. 14C, there are 4 struts and 4 cauterizing elements, although in other embodiments there may be less or more strut and cauterizing pairs, such as 3, 5, 6, 7, or 8.

FIG. 14C depicts a schematic, side view of capsule shell 656 being retracted to a second retracted position to expand struts 658 into the stent graft wall. Cauterizing elements 660 also contact the stent graft wall in this position because cauterizing elements 660 are affixed to struts 658. Cauterizing elements 660 are configured to be activated using a cutting mechanism (e.g., RF or resistance) to create incisions in the wall of the stent graft. An RF mechanism is configured to cut through the graft wall using vibrational forces. As shown in FIG. 14C, the incisions are 4 equal incisions in the wall of the stent graft.

FIG. 14D depicts a schematic, side view of capsule shell 656 being advanced back over struts 658 and cauterizing elements 660 thereby disengaging cauterizing elements 660. As shown in FIG. 14D, fenestration 662 formed by fenestration device 651 is diamond shaped, with the walls of the diamond optionally having a concave shape.

FIG. 14E depicts a schematic, side view of fenestration device 651 being retracted through guide catheter 654 leaving the diamond shaped fenestration 662 in the stent wall. Other non-limiting examples of shapes that may be formed by fenestration device 651 include a star shape and a plus sign shape.

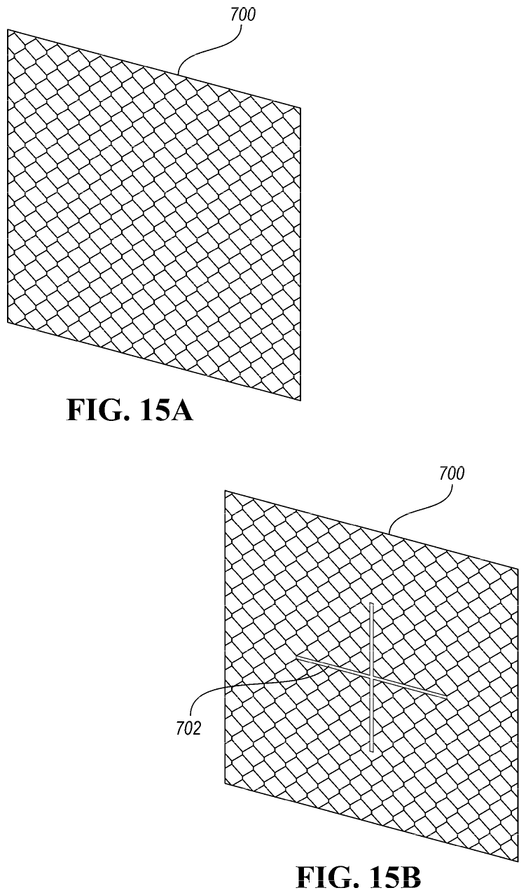

FIG. 15A depicts an isolated, perspective view of stent graft fabric 700 before a cauterization step. FIG. 15B depicts an isolated, perspective view of stent graft fabric 700 after the cauterization step. One or more embodiments use a catheter with a distal cauterization mechanism configured to create slotted plus cut 702 in stent graft fabric 700. The distal cauterization mechanism may include an adjustment mechanism to allow different diameter fenestrations. The distal cauterization mechanism may include a hot wire configured to cauterize the stent graft fabric and help prevent or mitigate particulate formation and/or fraying. After creating the fenestration, a side branch stent graft pushes aside and captures the quadrants of the fenestration.

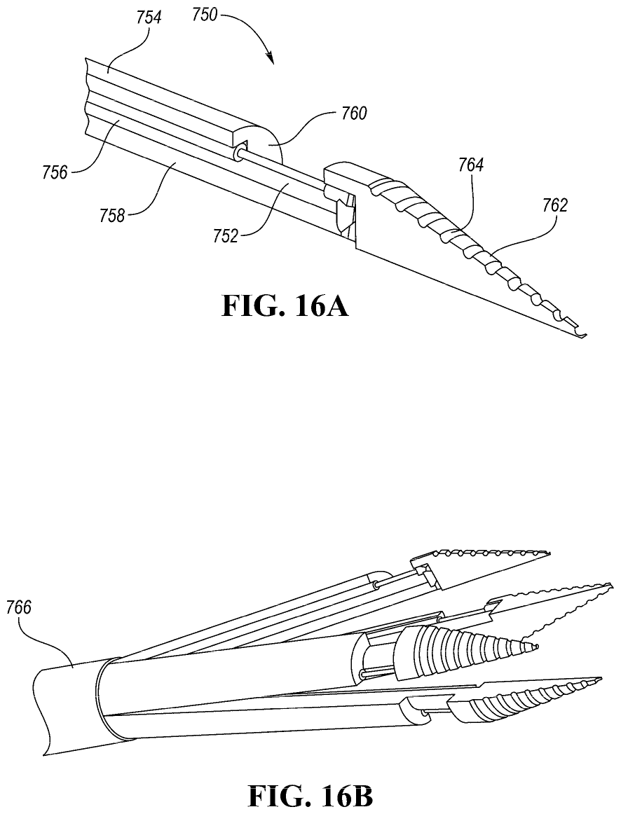

FIG. 16A depicts a perspective view of a portion of catheter cauterization device 750. FIG. 16A shows cauterization element 752. Catheter cauterization device 750 may be made up of 4 or more cauterization elements such as cauterization element 752. Each cauterization element may be built onto a frame (e.g., frame 754). The frame may be formed of nitinol or other shape memory element. The wire assembly (e.g., electrode wires 756 and 758) may be assembled onto the frame. Electrode wire 756 may be an anode wire and electrode wire 758 may be a cathode wire, or vice versa. The frame also includes proximal stop 760 configured to stop forward advancement of distal tip 762 relative to the operator. As shown in FIG. 16A, distal tip 762 is conical shaped with its apex at the distal end thereof and helical threads 764 configured to aid in cutting an initial hole at a fenestration site.

FIG. 16B depicts a perspective view of 4 cauterization elements returning to their open shape memory configuration once capsule shell 766 is retracted. By controlling the open position of capsule shell 766, the length of the cuts can be variable. For example, opening the capsule shell 50% may result in a 3 mm wide slot while opening the capsule shell 100% may result in a 6 mm wide slot. As another example, opening the capsule shell 75% may result in a 4.5 mm wide slot. The open position percentage is measured as the distance between a closed position where the capsule shell is closed such that the cauterization elements are compressed (e.g., compressed to not assume their shape memory shape) and a fully, opened position where the cauterization elements are fully in their shape memory shape. In between the fully closed and fully open positions, the distal end of the capsule shell biases the cauterization elements away from its fully open, shape memory shape.

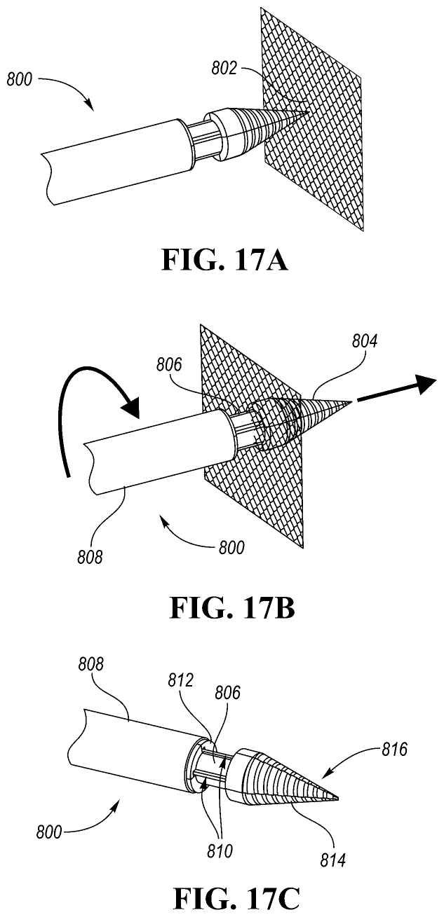

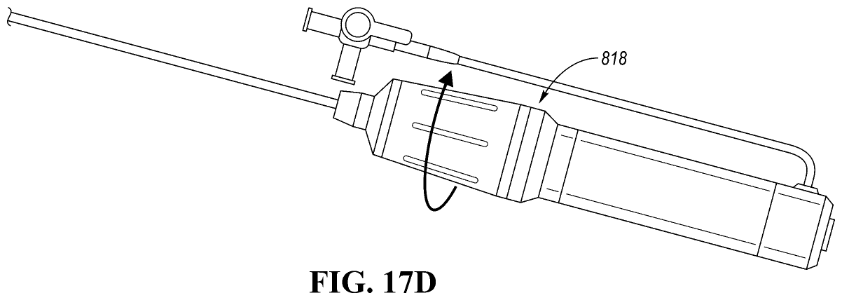

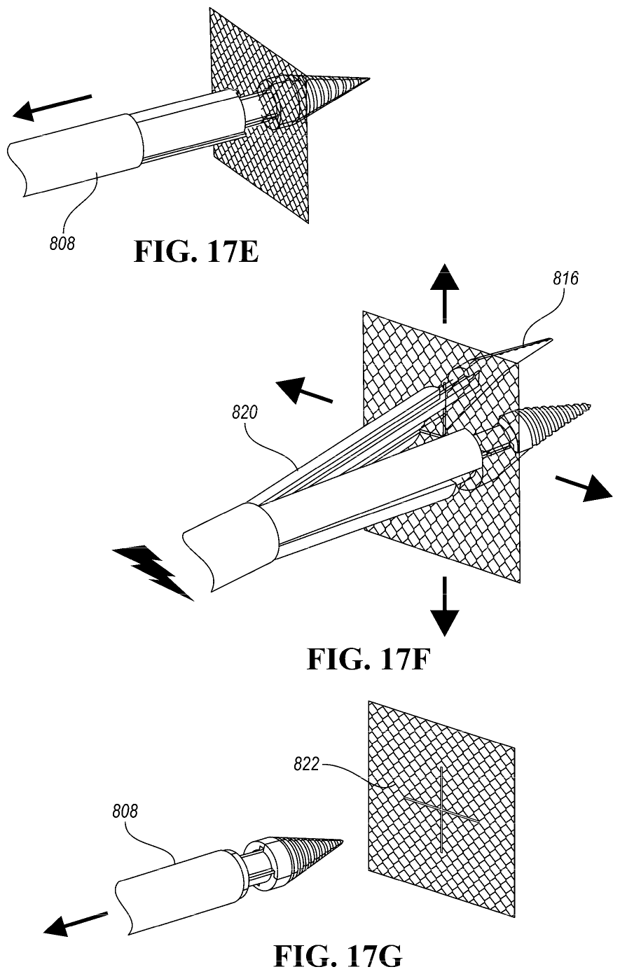

FIG. 17A depicts a perspective view of introducing fenestration device 800 through a guide catheter up to fenestration site 802. FIG. 17B depicts a perspective view of screwing or rotating distal tip 804 of fenestration device 800 into the graft material at fenestration site 802 until the graft material drops into slot 806 situated between distal tip 804 and capsule shell 808 to anchor the graft material for the subsequent cutting operation. Distal tip 804 includes helical threads to create a track for the rotational and distally advancing movement of fenestration device 800.

During delivery of fenestration device 800 to fenestration site 802, the cauterization elements are contained at least partially within capsule shell 808 to permit low profile tracking to fenestration site 802. FIG. 17C depicts a magnified, perspective view of fenestration device 800 include cauterization elements 810, capsule shell 808, and proximal stop 812. Helical threads 814 on cone-shaped distal tip 816 are configured to screw fenestration device 800 into the graft material. Slot 806 is configured to position the cauterization elements in contact with the graft material and act as an axial stop.

FIG. 17D depicts a perspective view of handle 818 of fenestration device 800 including fenestration diameter markers. As shown in FIG. 17D, handle 818 is configured to be rotated by a clinician to indicate the diameter of the fenestration (e.g., length of crosscuts of a plus signed shaped fenestration). Rotating handle 818 withdraws capsule shell 808 a distance to set the position of the cauterizing elements to obtain the indicated diameter. For instance, the clinician may set the diameter of handle 818 at 3 mm, which withdraws capsule shell 808 50% between the open and closed positions, thereby providing a three 3 mm diameter of the fenestration. FIG. 17E depicts a perspective view of capsule shell 808 retracting in a proximal direction from a closed position toward an open position in response to actuation (e.g., rotation) of handle 818.

FIG. 17F depicts a perspective view of cutter arms 820 in a partially open configuration. The clinician actuates electrical power to the cauterizing elements which rapidly heat up and cauterize the fabric to create a plus-shaped fenestration. Once the cauterization starts, the cauterizing elements can open and come to the partially open (or completely open) configuration determined by the capsule shell position (which in turn is determined by the handle position set by the clinician). The four cutter arms are pre-shaped to expand outward to cover the fenestration diameter range selectable through rotation of the handle. Cutter arms can be made of pre-shaped nitinol and delivered to a desired anatomical position with a capsule shell to maintain a low profile for delivery and retraction.

FIG. 17G depicts a perspective view of capsule shell 808 in a closed position configured to capture the cutter arms. Once the capsule shell is closed, the fenestration device is withdrawn leaving a plus shaped fenestration 822 in the graft material and configured to receive a branch stent graft. Upon withdrawal through the fenestration, the cutting wires are collapsed to their original, delivery size. This reduces the risk of the cutting wires catching on the graft material. In an alternative embodiment, the guide catheter is advanced to recapture the capsule and the helical threads prior to withdrawing the fenestration device through the fenestrated graft material.

The detailed description set forth herein includes several embodiments where each of the embodiments includes several components, features, and/or steps. For the avoidance of doubt, any component, feature, and/or step of one embodiment may be applied, mixed, substituted, matched, and/or combined with one or more components, features, and/or steps of other embodiments. Such resulting embodiments are expressly within the scope of this disclosure. For example, any systems and methods for locating a branch ostium of a branch vessel disclosed herein may be used in conjunction with any disclosed embodiments. Similarly, any systems, methods, or energy types for creating a fenestration (e.g., heat, laser, vibration, RF energy, blades/mechanical cutting) may be used in any disclosed embodiments. In any of the embodiments disclosed herein, following the creation of a fenestration the fenestration may be reinforced or strengthened by placing a stent or grommet like device in the fenestration. After a fenestration is created (and optionally reinforced), a branch stent graft may be tracked and deployed within the fenestration using a separate delivery system. The branch stent graft may extend within the fenestration and at least partially within a main lumen of the fenestrated stent graft and into branch artery (e.g., renal artery, celiac, SMA, BCA, LCC, LSA, etc.). The systems, methods, and devices disclosed herein may be used to make multiple fenestrations in a single stent graft, which thereafter each receive a branch stent graft.

While exemplary embodiments are described above, it is not intended that these embodiments describe all possible forms. The words used in the specification are words of description rather than limitation, and it is understood that various changes can be made without departing from the spirit and scope of the disclosure. As previously described, the features of various embodiments can be combined to form further embodiments of the invention that may not be explicitly described or illustrated. While various embodiments could have been described as providing advantages or being preferred over other embodiments or prior art implementations with respect to one or more desired characteristics, those of ordinary skill in the art recognize that one or more features or characteristics can be compromised to achieve desired overall system attributes, which depend on the specific application and implementation. These attributes can include, but are not limited to cost, strength, durability, life cycle cost, marketability, appearance, packaging, size, serviceability, weight, manufacturability, ease of assembly, etc. As such, to the extent any embodiments are described as less desirable than other embodiments or prior art implementations with respect to one or more characteristics, these embodiments are not outside the scope of the disclosure and can be desirable for particular applications.

What is claimed is:

1. A device for creating an in-situ fenestration in a graft material of a stent graft at a fenestration site, the cutting guidewire device comprising:
an outer member forming a lumen; and
an inner member situated within the lumen of the outer member,
the outer member includes one or more cutters configured to change from a delivery position to a cutting position via axial movement of the outer member and/or the inner member, the one or more cutters in the cutting position are configured to create the in-situ fenestration in the graft material of the stent graft at the fenestration site.

2. The device of claim 1, wherein the outer member includes one or more struts carrying the one or more cutters.

3. The device of claim 2, wherein the one or more cutters are one or more electrodes.

4. The device of claim 2, wherein the one or more cutters are one or more blades.

5. The device of claim 4, wherein the one or more blades are friction fit to the one or more struts.

6. The device of claim 4 further comprising an outer sheath configured to cover the one or more blades when the outer member is in the delivery position.

7. The device of claim 1, wherein the inner member is an inner wire including a distal tip configured to pierce the graft material at the fenestration site.

8. The device of claim 7, wherein the inner member includes a retaining shoulder and the outer member includes a proximal stop and a distal stop, the inner member is in a delivery position when the retaining shoulder contacts the proximal stop, and the inner member is configured to change into a deployment position.

9. The device of claim 8, wherein the distal tip extends beyond the outer member in the deployment position.

10. The device of claim 8, wherein the retaining shoulder contacts the distal stop when the inner member is in the deployment position.

11. A device for creating an in-situ fenestration in a graft material of a stent graft at a fenestration site, the cutting guidewire device comprising:
an outer member including a proximal end and a distal end and one or more struts extending therebetween, the one or more struts carrying one or more cutters; and
an inner member situated within the outer member,
the one or more struts are configured to change from a delivery position to a cutting position via axial movement of the outer member and/or the inner member, the one or more cutters in the cutting position are configured to create the in-situ fenestration in the graft material of the stent graft at the fenestration site, the one or more cutters are spaced apart from the inner member in the cutting position.

12. The device of claim 11, wherein the one or more struts are bent in the cutting position.

13. The device of claim 12, wherein the one or more struts include one or more tapered portions.

14. The device of claim 11, wherein the one or more struts are linear in the delivery position.

15. The device of claim 11, wherein the proximal end includes a transverse cutting pattern configured for flexing in the delivery position.

16. A method of deploying a device at a fenestration site to form a fenestration in a graft material of a stent graft, the method comprises:
delivering a distal tip of the cutting guidewire device to the fenestration site;
penetrating the graft material of the stent graft at the fenestration site with the distal tip; and
creating the fenestration at the fenestration site with one or more cutters of the cutting guidewire device.

17. The method of claim 16, wherein the cutting guidewire device includes an outer member including one or more struts carrying the one or more cutters and an inner member situated within the outer member.

18. The method of claim 17 further comprising aligning the one or more cutters with the graft material, compressing the outer member to urge the one or more struts carrying the one or more cutters outward, and activating the one or more cutters to create the fenestration.

19. The method of claim 18, wherein the one or more cutters are one or more electrodes, and the activating step includes energizing the one or more electrodes to create the fenestration.

20. The method of claim 18 further comprising retracting the distal tip into the outer member after the aligning step.
